(19) United States
(12) Patent Application Publication
(10) Pub. No.: US 2007/0027050 A1
(43) Pub. Date: Feb. 1, 2007

(54) Liquid cleansing composition

(75) Inventors: Brian Andrew Crotty, Branford, CT (US); Jochen Weiss, Stratford, CT (US); Suzanne Lynn Gencarelli, Milford, CT (US); Shauna Mary Lagatol, New Haven, CT (US); Nicolas E. Restrepo, West Haven, CT (US); Virgilio Barba Villa, Emerson, NJ (US); Craig Stephen Slavtcheff, Guilford, CT (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Conopco, Inc., d/b/a UNILEVER

(21) Appl. No.: 11/190,599

(22) Filed: Jul. 27, 2005

(51) Int. Cl. A61K 8/00

(52) U.S. Cl. 510/130

(57) ABSTRACT

A mild, aqueous, liquid crystalline structured cleansing and moisturizing composition with low levels of surfactants that include monoalkyl sulfosuccinate surfactant(s) and high levels of hydrophillic emollients is provided that provides a low in-use slip, tight skin feel, and a fast, clean-feel rinse in the absence of substantial levels of soap. Substantial amounts of persistent and fine quality lather are produced with the inventive composition even in the presence of high levels of emollients.

### BACKGROUND OF THE INVENTION

1. Field of the Invention

The present invention relates to detergent compositions suitable for topical application for cleansing the human body, such as the skin and hair. In particular, it relates to stable liquid cleansing compositions containing a specific combination of polyol(s), and low levels of monoalkyl sulfosuccinate(s) and optionally other non-soap surfactants that have advantageous sensory properties.

2. Background of the Art

Prior art skin cleansers modify the way the skin feels after the shower by depositing materials such as oils or polymers. However, such cleansers often have disadvantageous sensory or physical properties such as a slimy feel and/or poor lather. Stability problems are observed with other combinations of hydrophilic emollients and surfactants.

U.S. Pat. No. 5,635,469 issued to Fowler et al. on Jun. 3, 1997 discloses a cleansing composition with up to 25% by wt. of hydrophillic emollients. U.S. Pat. No. 5,312,559 issued to Kacher et al. on May 17, 1994 discloses a cleansing composition containing substantial levels of both soap and hydrophillic emollients. U.S. Pat. No. 4,812,253 issued to Small et al. on Mar. 14, 1989 also discloses a cleansing composition containing substantial levels of both soap and hydrophillic emollients. U.S. Pat. No. 6,113,892 issued to Newell et al on Sep. 5, 2000 discloses a cleansing composition with low levels of hydrophillic emollients up to about 5% by wt.

EP Patent 1235890 titled Stable, High Glycerol Liquids Comprising N-Acyl Amino Acids and/or Salts and issued to Arai et al. discloses high content glycerol liquid compositions comprising N-acyl amino acids and/or salts and defined sulfosuccinic acid monoesters and a method of enhancing the stability of high content glycerin compositions comprising N-acyl amino acids or salts thereof and sulfosuccinic acid monoesters.

Surprisingly it has been discovered that by incorporating specific emollients and non-soap surfactants in specific amounts in a cleanser formulation, excellent sensory and lather properties can be obtained simultaneously with enhanced mildness.

### BRIEF DESCRIPTION OF THE INVENTION

In one aspect of the invention is a cleansing composition including but not limited to:
a. at least about 20% by wt. of total polyol(s)
b. at least about 3% by wt. of total normal C6 to C16 alkyl monosulfosuccinate(s);
c. at least about 4% by wt. of vegetable derived glyceride oil(s);
d. at least about 0.1% by wt. of mineral oil(s);
e. a maximum of about 5% by wt. of soap;
f. at least about 15% by wt. of water;
g. an effective amount of an ordered liquid crystalline structurant for forming an ordered liquid crystalline phase in said composition;
h. a viscosity in the range of about 5,000 to 100,000,000 cps at 25 C using the t-bar method. and
i. wherein said composition has a lather type attribute of −1 or less and an ease of rinse attribute of −1 or greater according the Trained Panel Test.

### BRIEF DESCRIPTION OF THE DRAWINGS

FIG. 1 is a graphical representation comparing the rinse and lather profiles as defined in the Panel Test Method of two preferred embodiments of the inventive composition to an example of a comparative composition.

FIG. 2 is a graphical representation comparing the tightness skin quality and skin feel profiles as defined in the Panel Test Method of two preferred embodiments of the inventive composition to an example of a comparative composition.

### DETAILED DESCRIPTION OF THE INVENTION

All publications and patent applications, patents, and other references mentioned herein are incorporated by reference in their entirety.

In one aspect of the invention is a cleansing composition including but not limited to:
a. at least about 20% by wt. of total polyol(s); preferably at least about 25, 30, 35 or 40%; preferably a maximum of about 60% by wt.
b. at least about 3% by wt. of total normal C6 to C16 alkyl monosulfosuccinate(s); preferably C10 to C16 alkyl and more preferably in the concentration range of about 3 to 10% by wt.
c. at least about 4% by wt. of vegetable derived glyceride oil(s); preferably at least about 10, 15 or 20% by wt.; and preferably the maximum is about 25 or 30% by wt.
d. at least about 0.1% by wt. of mineral oil(s); preferably at least about 1, 3, 5%; and preferably the maximum is about 10, 15, or 20% by wt.;
e. a maximum of 5% by wt. of soap; preferably less than 4, 3, 2, 1, 0.5, 0.2 or 0.1% by wt. of soap;
f. at least about 15% by wt. of water; preferably in the range of about 15 to 95% by wt.; and more preferably in the range of about 30 to 70% by wt.
g. an effective amount of an ordered liquid crystalline structurant for forming an ordered liquid crystalline phase in said composition; preferably in the concentration range of about 0.1 to 15% by wt. and preferably selected from lauric acid;
h. a viscosity in the range of about 5,000 to 100,000,000 cps at 25 C using the t-bar method. and
i. wherein said composition has a lather type attribute of −1 or less and an ease of rinse attribute of −1 or greater according the Trained Panel Test.

Advantageously, the inventive composition has a skin tightness attribute of 0.5 or greater and a skin feel attribute of −0.2 or greater according to the Trained Panel Test. Preferably the total surfactant content is below a maximum of about 15% by wt. More preferably the ratio of total polyol(s) to total glyceride and mineral oils is in the range of about 1:40 to about 1:4; advantageously in the range of about 1:10 to about 1:20.

Advantageously, the ratio of total glyceride and mineral oils) to total surfactant(s) is in the range of about 1:10 to about 10:1; preferably in the range of about 1:2 to about 2:1. Preferably the ratio of total mono and disulfosuccinate(s) to total polyol(s) is in the range of about 1:4 to about 1:2; more preferably in the range of about 1:10 to about 1:20. Advantageously the polyol(s) is/are selected from glycerin, diglycerin, ethoxylated glycerin, propoxylated glycerin, propylene glycol, dipropylene glycol, polypropylene glycol Polyethylene glycol, sorbitol, hydroxylpropyl sorbitol, hexylene glycol, 1,3-butylene glycol and 1,2,6 hexanetriol and blends thereof and the like.

Advantageously the monosulfosuccinate(s) is/are selected from C10, C12, or C14 and blends thereof; the vegetable derived glyceride oil(s) is/are selected from sunflower seed oil, soybean oil, jojoba oil, canola oil, coconut oil, peanut oil, or palm oil and blends thereof and the like and the mineral oil(s) is/are selected from petrolatum, mineral oil, heavy mineral oil, parafin wax, and blends thereof and the like.

In another preferred embodiment the inventive composition includes cationic polymer(s) in a concentration range of about 0.01% to 2.0% wt. %, advantageously selected from polyquaternium 55, polyquaternium 7, polyqaternium 39, polyquat 10, and blends thereof and the like. Preferably the composition has a lamellar structure, and in a further preferred aspect, the composition further includes an effective concentration of an active ingredient selected from anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sunscreen actives, sebum stimulators, sebum inhibitors, antiperspirants, anti-glycation actives or mixtures thereof.

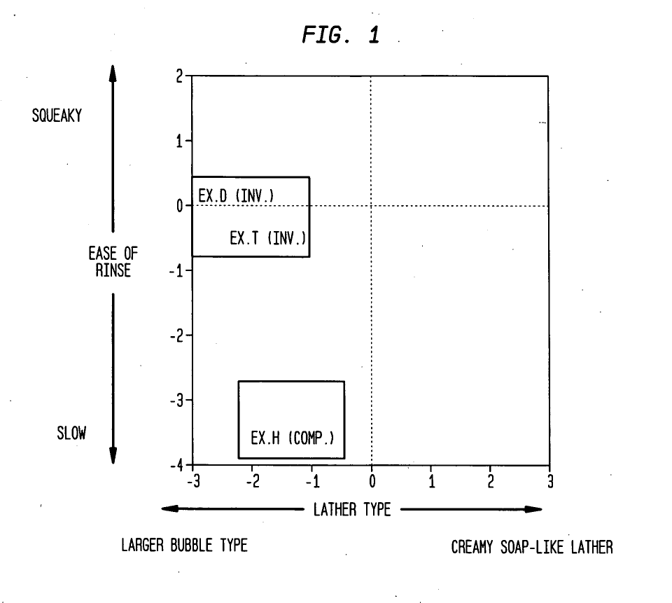

Referring to FIG. 1, the rinse and lather profiles of inventive examples D and T and comparative example H as described in Table 1 are graphically depicted. The rinse and lather profile data are tabulated in Table 1 and were determined by a trained panel according to the Panel Testing procedure described below.

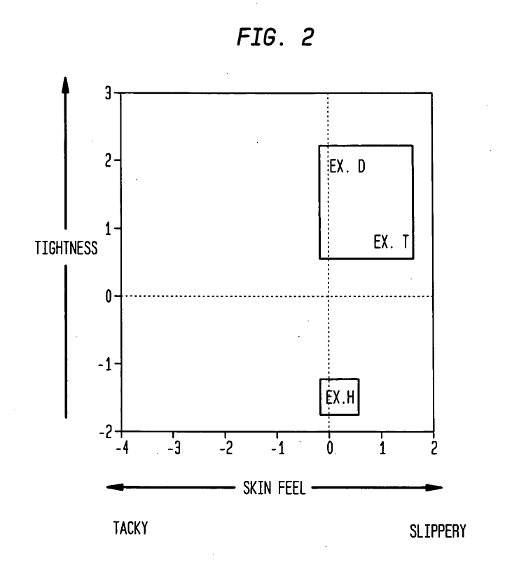

Now referring to FIG. 2, the tightness skin quality and skin feel profiles of inventive examples D and T and comparative example H are graphically depicted and are defined and measured according to the Panel Testing procedure. The tightness and skin feel profile data are tabulated in Table 1.

Surfactants:

Surfactants are an essential component of the inventive cleansing composition. They are compounds that have hydrophobic and hydrophilic portions that act to reduce the surface tension of the aqueous solutions they are dissolved in. In addition to the surfactants required by the invention, other useful surfactants can be added to the inventive composition and can include anionic, nonionic, amphoteric, and cationic surfactants, and blends thereof.

Anionic Surfactants:

The cleansing composition of the present invention contains monoalkyl sulfosuccinate(s) (e.g., C₆-C₂₂ sulfosuccinates) and optionally other anionic surfactants. Preferably the monoalkyl sulfosuccinates are used in the range of about 0.5% to 15% by wt. and preferably in the range of about 3 to 10% by wt.

Monoalkyl Sulfosuccinates Having the Formula:

R⁴O₂CCH₂CH(SO₃M)CO₂M; are usefully employed in the invention as described above wherein R⁴ ranges from C₁₀-C₁₆ alkyl and M is a solubilizing cation.

Other anionic detergent actives which may be used include aliphatic sulfonates, such as a primary alkane (e.g., C₈-C₂₂) sulfonate, primary alkane (e.g., C₈-C₂₂) disulfonate, C₈-C₂₂ alkene sulfonate, C₈-C₂₂ hydroxyalkane sulfonate or alkyl glyceryl ether sulfonate (AGS); or aromatic sulfonates such as alkyl benzene sulfonate.

The anionic may also be an alkyl sulfate (e.g., C₁₂-C₁₈ alkyl sulfate) or alkyl ether sulfate (including alkyl glyceryl ether sulfates). Among the alkyl ether sulfates are those having the formula:
RO(CH₂CH₂O)ₙSO₃M
wherein R is an alkyl or alkenyl having 8 to 18 carbons, preferably 12 to 18 carbons, n has an average value of greater than 1.0, preferably greater than 3; and M is a

solubilizing cation such as sodium, potassium, ammonium or substituted ammonium. Ammonium and sodium lauryl ether sulfates are preferred.

The anionic may also include dialkyl sulfosuccinates (e.g., C₆-C₂₂ sulfosuccinates); alkyl and acyl taurates, alkyl and acyl sarcosinates, sulfoacetates, C₈-C₂₄ monoalkyl or dialkyl phosphates, n-acyl amino acid surfactant(s) alkyl phosphate esters and alkoxyl alkyl phosphate esters, acyl lactates, C₈-C₂₂ monoalkyl succinates and maleates, sulphoacetates, alkyl glucosides and acyl isethionates, and the like.

Amide-MEA sulfosuccinates of the formula;
R⁴CONHCH₂CH₂O₂CCH₂CH(SO₃M)CO₂M

May be used wherein R⁴ ranges from C₈-C₂₂ alkyl and M is a solubilizing cation may be used.

Sarcosinates are generally indicated by the formula:
R¹CON(CH₃)CH₂CO₂M,

wherein R¹ ranges from C₈-C₂₀ alkyl and M is a solubilizing cation.

Taurates are generally identified by formula:
R²CONR³CH₂CH₂SO₃M

wherein R² ranges from C₈-C₂₀ alkyl, R³ ranges from C₁-C₄ alkyl and M is a solubilizing cation.

The inventive cleansing composition may contain C₈-C₁₈ acyl isethionates. These esters are prepared by reaction between alkali metal isethionate with mixed aliphatic fatty acids having from 6 to 18 carbon atoms and an iodine value of less than 20. At least 75% of the mixed fatty acids have from 12 to 18 carbon atoms and up to 25% have from 6 to 10 carbon atoms.

The acyl isethionate may be an alkoxylated isethionate such as is described in Ilardi et al., U.S. Pat. No. 5,393,466, titled “Fatty Acid Esters of Polyalkoxylated isethonic acid; issued Feb. 28, 1995; hereby incorporated by reference. This compound has the general formula:
R C—O(O)—C(X)H—C(Y)H₂—(OCH—CH₂)ₘ—SO₃M⁺

wherein R is an alkyl group having 8 to 18 carbons, m is an integer from 1 to 4, X and Y are hydrogen or an alkyl group having 1 to 4 carbons and M⁺ is a monovalent cation such as, for example, sodium, potassium or ammonium.

Amphoteric Surfactants

One or more amphoteric surfactants may be used in this invention. Amphoteric surfactants are preferably used at levels as low as 0.5, 1, 2, 3, 4 or 5% by wt. and at levels as high as 6, 8, 10, 12, or 15% by wt. Such surfactants include at least one acid group. This may be a carboxylic or a sulphonic acid group. They include quaternary nitrogen and therefore are quaternary amido acids. They should generally include an alkyl or alkenyl group of 7 to 18 carbon atoms. They will usually comply with an overall structural formula:
R¹—[—C(O)—NH (CH₂)ₙ—]ₘ—N⁺—(R²)(R³)X—Y
where R¹ is alkyl or alkenyl of 7 to 18 carbon atoms;
R² and R³ are each independently alkyl, hydroxyalkyl or carboxyalkyl of 1 to 3 carbon atoms;
n is 2 to 4;
m is 0 to 1;
X is alkylene of 1 to 3 carbon atoms optionally substituted with hydroxyl, and
Y is —CO₂— or —SO₃—

Suitable amphoteric surfactants within the above general formula include simple betaines of formula:
R¹—N⁺—(R²)(R³)CH₂CO₂⁻
and amido betaines of formula:
   R¹—CONH(CH₂)ₙ—N⁺—(R²)(R³)CH₂CO₂⁻
where n is 2 or 3.

In both formulae R¹, R² and R³ are as defined previously. R¹ may in particular be a mixture of C₁₂ and C₁₄ alkyl groups derived from coconut oil so that at least half, preferably at least three quarters of the groups R¹ have 10 to 14 carbon atoms. R² and R³ are preferably methyl.

A further possibility is that the amphoteric detergent is a sulphobetaine of formula:
R¹—N⁺—(R²)(R³) (CH₂)₃SO₃⁻
or
R¹—CONH(CH₂)ₘ—N⁺—(R²)(R³) (CH₂)₃SO₃⁻
where m is 2 or 3, or variants of these in which —(CH₂)₃SO₃⁻ is replaced by
   —CH₂C(OH)(H)CH₂SO₃⁻
In these formulae R¹, R² and R³ are as discussed previously.

Amphoacetates and diamphoacetates are also intended to be covered in possible zwitterionic and/or amphoteric compounds which may be used such as e.g., sodium lauroamphoacetate, sodium cocoamphoacetate, and blends thereof, and the like.

Nonionic Surfactants

One or more nonionic surfactants may also be used in the cleansing composition of the present invention. Nonionic surfactants are preferably used at levels as low as 0.5, 1, 2, 3 or 5% by wt. and at levels as high as 6, 8, 10, 12 or 15% by wt. The nonionics which may be used include in particular the reaction products of compounds having a hydrophobic group and a reactive hydrogen atom, for example aliphatic alcohols, acids, amides or alkylphenols with alkylene oxides, especially ethylene oxide either alone or with propylene oxide. Specific nonionic detergent compounds are alkyl (C₆-C₂₂) phenols ethylene oxide condensates, the condensation products of aliphatic (C₈-C₁₈) primary or secondary linear or branched alcohols with ethylene oxide, and products made by condensation of ethylene oxide with the reaction products of propylene oxide and ethylenediamine. Other so-called nonionic detergent compounds include long chain tertiary amine oxides, long chain tertiary phosphine oxides and dialkyl sulphoxide, and the like.

The nonionic may also be a sugar amide, such as a polysaccharide amide. Specifically, the surfactant may be one of the lactobionamides described in U.S. Pat. No. 5,389,279 to Au et al. titled “Compositions Comprising Nonionic Glycolipid Surfactants issued Feb. 14, 1995; which is hereby incorporated by reference or it may be one of the sugar amides described in U.S. Pat. No. 5,009,814 to Kelkenberg, titled “Use of N-Poly Hydroxyalkyl Fatty Acid Amides as Thickening Agents for Liquid Aqueous Surfactant Systems” issued Apr. 23, 1991; hereby incorporated into the subject application by reference.

Cationic Skin Conditioning Agents

A useful component in compositions according to the invention is a cationic skin feel agent or polymer, such as for example cationic celluloses. Cationic polymers are preferably used at levels as low as about 0.01, 0.05, 0.1, 0.5, 1 or 2% and at levels as high as about 2, 3, 4 or 5% by wt. Cationic cellulose is available from Amerchol Corp. (Edison, N.J., USA) in their Polymer JR (trade mark) and LR (trade mark) series of polymers, as salts of hydroxyethyl cellulose reacted with trimethyl ammonium substituted epoxide, referred to in the industry (CTFA) as Polyquaternium 10. Another type of cationic cellulose includes the polymeric quaternary ammonium salts of hydroxyethyl cellulose reacted with lauryl dimethyl ammonium-substituted epoxide, referred to in the industry (CTFA) as Polyquaternium 24. These materials are available from Amerchol Corp. (Edison, N.J., USA) under the tradename Polymer LM-200.

A particularly suitable type of cationic polysaccharide polymer that can be used is a cationic guar gum derivative, such as guar hydroxypropyltrimonium chloride (Commercially available from Rhone-Poulenc in their JAGUAR trademark series). Examples are JAGUAR C13S, which has a low degree of substitution of the cationic groups and high viscosity, JAGUAR C15, having a moderate degree of substitution and a low viscosity, JAGUAR C17 (high degree of substitution, high viscosity), JAGUAR C16, which is a hydroxypropylated cationic guar derivative containing a low level of substituent groups as well as cationic quaternary ammonium groups, and JAGUAR 162 which is a high transparency, medium viscosity guar having a low degree of substitution.

Particularly preferred cationic polymers are JAGUAR C13S, JAGUAR C15, JAGUAR C17 and JAGUAR C16 and JAGUAR C162, especially Jaguar C13S. Other cationic skin feel agents known in the art may be used provided that they are compatible with the inventive formulation.

Cationic Surfactants

One or more cationic surfactants may also be used in the cleansing composition. Cationic surfactants may be used at levels as low as about 0.01, 0.05, 0.1, 0.5, 1% by wt. and at levels as high as 2, 3, 4 or 5% by wt.

Examples of cationic detergents are the quaternary ammonium compounds such as alkyldimethylammonium halogenides. Other suitable surfactants which may be used are described in U.S. Pat. No. 3,723,325 to Parran Jr. titled “Detergent Compositions Containing Particle Deposition Enhancing Agents” issued Mar. 27, 1973; and “Surface Active Agents and Detergents” (Vol. I & II) by Schwartz, Perry & Berch, both of which are also incorporated into the subject application by reference.

In addition, the inventive cleansing composition of the invention may include 0 to 15% by wt. optional ingredients as follows: perfumes; sequestering agents, such as tetrasodium ethylenediaminetetraacetate (EDTA), EHDP or mixtures in an amount of 0.01 to 1%, preferably 0.01 to 0.05%; and coloring agents, opacifiers and pearlizers such as zinc stearate, magnesium stearate, TiO₂, EGMS (ethylene glycol monostearate) or Lytron 621 (Styrene/Acrylate copolymer) and the like; all of which are useful in enhancing the appearance or cosmetic properties of the product.

The compositions may further comprise antimicrobials such as 2-hydroxy-4,2′, 4′ trichlorodiphenylether (DP300); preservatives such as dimethyloldimethylhydantoin (Glydant XL 1000), parabens, sorbic acid etc., and the like.

The compositions may also comprise coconut acyl mono- or diethanol amides as suds boosters, and strongly ionizing salts such as sodium chloride and sodium sulfate may also be used to advantage.

Antioxidants such as, for example, butylated hydroxytoluene (BHT) and the like may be used advantageously in amounts of about 0.01% or higher if appropriate.

Moisturizers (also known as hydrophilic emollients) that also are Humectants such as polyhydric alcohols, e.g. glycerine and propylene glycol, and the like; and polyols such as the polyethylene glycols listed below and the like are used as described above.

| | |
|---|---|
| Polyox WSR-205 | PEG 14M, |
| Polyox WSR-N-60K | PEG 45M, or |
| Polyox WSR-N-750 | PEG 7M. |

Vegetable derived glyceride oil(s) and mineral oil(s) as mentioned above are hydrophobic emollients that are used in the invention. Other hydrophobic emollients may be used at levels that do not alter the unique sensory properties of the invention.

The term “emollient” (also considered skin conditioning compounds according to the invention) is defined as a substance which softens or improves the elasticity, appearance, and youthfulness of the skin (stratum corneum) by either increasing its water content, adding, or replacing lipids and other skin nutrients; or both, and keeps it soft by retarding the decrease of its water content.

Useful hydrophobic emollients include the following:

(a) silicone oils and modifications thereof such as linear and cyclic polydimethylsiloxanes; amino, alkyl, alkylaryl, and aryl silicone oils;

(b) fats and oils including natural fats and oils such as jojoba, soybean, sunflower, rice bran, avocado, almond, olive, sesame, persic, castor, coconut, mink oils; cacao fat; beef tallow, lard; hardened oils obtained by hydrogenating the aforementioned oils; and synthetic mono, di and triglycerides such as myristic acid glyceride and 2-ethylhexanoic acid glyceride;

(c) waxes such as carnauba, spermaceti, beeswax, lanolin, and derivatives thereof;

(d) hydrophobic and hydrophillic plant extracts;

(e) hydrocarbons such as liquid paraffins, vaseline, microcrystalline wax, ceresin, squalene, pristan and mineral oil;

(f) higher fatty acids such as lauric, myristic, palmitic, stearic, behenic, oleic, linoleic, linolenic, lanolic, isostearic, arachidonic and poly unsaturated fatty acids (PUFA);

(g) higher alcohols such as lauryl, cetyl, stearyl, oleyl, behenyl, cholesterol and 2-hexydecanol alcohol;

(h) esters such as cetyl octanoate, myristyl lactate, cetyl lactate, isopropyl myristate, myristyl myristate, isopropyl palmitate, isopropyl adipate, butyl stearate, decyl oleate, cholesterol isostearate, glycerol monostearate, glycerol distearate, glycerol tristearate, alkyl lactate, alkyl citrate and alkyl tartrate;

(i) essential oils and extracts thereof such as mentha, jasmine, camphor, white cedar, bitter orange peel, ryu, turpentine, cinnamon, bergamot, citrus unshiu, calamus, pine, lavender, bay, clove, hiba, eucalyptus, lemon, starflower, thyme, peppermint, rose, sage, sesame, ginger, basil, juniper, lemon grass, rosemary, rosewood, avocado, grape, grapeseed, myrrh, cucumber, watercress, calendula, elder flower, geranium, linden blossom, amaranth, seaweed, ginko, ginseng, carrot, guarana, tea tree, jojoba, comfrey, oatmeal, cocoa, neroli, vanilla, green tea, penny royal, aloe vera, menthol, cineole, eugenol, citral, citronelle, borneol, linalool, geraniol, evening primrose, camphor, thymol, spirantol, penene, limonene and terpenoid oils;

(j) mixtures of any of the foregoing components, and the like.

Ordered Liquid Crystalline Compositions:

The inventive cleansing composition possesses ordered liquid crystalline microstructure, preferably lamellar microstructure. The rheological behavior of all surfactant solutions, including liquid cleansing solutions, is strongly dependent on the microstructure, i.e., the shape and concentration of micelles or other self-assembled structures in solution.

When there is sufficient surfactant to form micelles (concentrations above the critical micelle concentration or CMC), for example, spherical, cylindrical (rod-like or discoidal), spherocylindrical or ellipsoidal micelles may form. As surfactant concentration increases, ordered liquid crystalline phases such as lamellar phase, hexagonal phase, cubic phase or L3 sponge phase may form. The lamellar phase, for example, consists of alternating surfactant bilayers and water layers. These layers are not generally flat but fold to form submicron spherical onion like structures called vesicles or liposomes. The hexagonal phase, on the other hand, consists of long cylindrical micelles arranged in a hexagonal lattice. In general, the microstructure of most personal care products consist of either spherical micelles; rod micelles; or a lamellar dispersion.

As noted above, micelles may be spherical or rod-like. Formulations having spherical micelles tend to have a low viscosity and exhibit Newtonian shear behavior (i.e., viscosity stays constant as a function of shear rate; thus, if easy pouring of product is desired, the solution is less viscous and, as a consequence, it doesn't suspend as well). In these systems, the viscosity increases linearly with surfactant concentration.

Rod micellar solutions are more viscous because movement of the longer micelles is restricted. At a critical shear rate, the micelles align and the solution becomes shear thinning. Addition of salts increases the size of the rod micelles thereof increasing zero shear viscosity (i.e., viscosity when sitting in bottle) which helps suspend particles but also increases critical shear rate (point at which product becomes shear thinning; higher critical shear rates means product is more difficult to pour).

Lamellar dispersions differ from both spherical and rod-like micelles because they can have high zero shear viscosity (because of the close packed arrangement of constituent lamellar droplets), yet these solutions are very shear thinning (readily dispense on pouring). That is, the solutions can become thinner than rod micellar solutions at moderate shear rates.

In formulating liquid cleansing compositions, therefore, there is the choice of using rod-micellar solutions (whose zero shear viscosity, e.g., suspending ability, is not very good and/or are not very shear thinning); or lamellar dispersions (with higher zero shear viscosity, e.g. better suspending, and yet are very shear thinning). Such lamellar compositions are characterized by high zero shear viscosity (good for suspending and/or structuring) while simultaneously being very shear thinning such that they readily dispense in pouring. Such compositions possess a “heaping”, lotion-like appearance which convey signals of enhanced moisturization.

When rod-micellar solutions are used, they also often require the use of external structurants to enhance viscosity and to suspend particles (again, because they have lower zero shear viscosity than lamellar phase solutions). For this, carbomers and clays are often used. At higher shear rates (as in product dispensing, application of product to body, or rubbing with hands), since the rod-micellar solutions are less shear thinning, the viscosity of the solution stays high and the product can be stringy and thick. Lamellar dispersion based products, having higher zero shear viscosity, can more readily suspend emollients and are typically more creamy. In general, lamellar phase compositions are easy to identify by their characteristic focal conic shape and oily streak texture while hexagonal phase exhibits angular fan-like texture. In contrast, micellar phases are optically isotropic.

It should be understood that lamellar phases may be formed in a wide variety of surfactant systems using a wide variety of lamellar phase “inducers” as described, for example, in U.S. Pat. No. 5,952,286 issued to Puvvada, et al., on Sep. 14, 1999. Generally, the transition from micelle to lamellar phase are functions of effective average area of headgroup of the surfactant, the length of the extended tail, and the volume of tail. Using branched surfactants or surfactants with smaller headgroups or bulky tails are also effective ways of inducing transitions from rod micellar to lamellar.

One way of characterizing ordered liquid crystalline dispersions include measuring viscosity at low shear rate (using for example a Stress Rheometer) when additional inducer (e.g., oleic acid or isostearic acid) is used. At higher amounts of inducer, the low shear viscosity will significantly increase.

Another way of measuring ordered liquid crystalline dispersions is using freeze fracture electron microscopy. Micrographs generally will show ordered liquid crystalline microstructure and close packed organization of the lamellar droplets (generally in size range of about 2 microns).

In a preferred embodiment, the inventive ordered liquid crystalline phase composition preferably has a low shear viscosity in the range of about 40,000 to about 300,000 centipoises (cps) measured at 0.5 RPM using T-bar spindle A using the procedure described below. More preferably the viscosity range is about 40,000 to about 150,000 cps.

Optional Active Agents

Advantageously, active agents other than conditioning agents such as emollients or moisturizers defined above may be added to the cleansing composition in a safe and effective amount during formulation to treat the skin during the use of the product. These active ingredients may be advantageously selected from antimicrobial and antifungal actives, vitamins, anti-acne actives; anti-wrinkle, anti-skin atrophy and skin repair actives; skin barrier repair actives; non-steroidal cosmetic soothing actives; artificial tanning agents and accelerators; skin lightening actives; sunscreen actives; sebum stimulators; sebum inhibitors; anti-oxidants; protease inhibitors; skin tightening agents; anti-itch ingredients; hair growth inhibitors; 5-alpha reductase inhibitors; desquamating enzyme enhancers; anti-glycation agents; topical anesthetics, or mixtures thereof; and the like.

These active agents may be selected from water soluble active agents, oil soluble active agents, pharmaceutically-acceptable salts and mixtures thereof. Advantageously the agents will be soluble or dispersible in the cleansing composition. The term “active agent” as used herein, means personal care actives which can be used to deliver a benefit to the skin and/or hair and which generally are not used to confer a conditioning benefit, as is conferred by humectants and emollients previously described herein. The term “safe and effective amount” as used herein, means an amount of active agent high enough to modify the condition to be treated or to deliver the desired skin care benefit, but low enough to avoid serious side effects. The term “benefit,” as used herein, means the therapeutic, prophylactic, and/or chronic benefits associated with treating a particular condition with one or more of the active agents described herein. What is a safe and effective amount of the active agent ingredient will vary with the specific active agent, the ability of the active to penetrate through the skin, the age, health condition, and skin condition of the user, and other like factors. Preferably the composition of the present invention comprise from about 0.01% to about 50%, more preferably from about 0.05% to about 25%, even more preferably 0.1% to about 10%, and most preferably 0.1% % to about 5%, by weight of the active agent component.

Anti-acne actives can be effective in treating acne vulgaris, a chronic disorder of the pilosebaceous follicles. Nonlimiting examples of useful anti-acne actives include the keratolytics such as salicylic acid (o-hydroxybenzoic acid), derivatives of salicylic acid such as 5-octanoyl salicylic acid and 4 methoxysalicylic acid, and resorcinol; retinoids such as retinoic acid and its derivatives (e.g., cis and trans); sulfur-containing D and L amino acids and their derivatives and salts, particularly their N-acetyl derivatives, mixtures thereof and the like.

Antimicrobial and antifungal actives can be effective to prevent the proliferation and growth of bacteria and fungi. Nonlimiting examples of antimicrobial and antifungal actives include b-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, triclosan; triclocarban; and mixtures thereof and the like.

Anti-wrinkle, anti-skin atrophy and skin repair actives can be effective in replenishing or rejuvenating the epidermal layer. These actives generally provide these desirable skin care benefits by promoting or maintaining the natural process of desquamation. Nonlimiting examples of antiwrinkle and anti-skin atrophy actives include vitamins, minerals, and skin nutrients such as milk, vitamins A, E, and K; vitamin alkyl esters, including vitamin C alkyl esters; magnesium, calcium, copper, zinc and other metallic components; retinoic acid and its derivatives (e.g., cis and trans); retinal; retinol; retinyl esters such as retinyl acetate, retinyl palmitate, and retinyl propionate; vitamin B 3 compounds (such as niacinamide and nicotinic acid), alpha hydroxy acids, beta hydroxy acids, e.g. salicylic acid and derivatives thereof (such as 5-octanoyl salicylic acid, heptyloxy 4 salicylic acid, and 4-methoxy salicylic acid); mixtures thereof and the like.

Skin barrier repair actives are those skin care actives which can help repair and replenish the natural moisture barrier function of the epidermis. Nonlimiting examples of skin barrier repair actives include lipids such as cholesterol, ceramides, sucrose esters and pseudo-ceramides as described in European Patent Specification No. 556,957; ascorbic acid; biotin; biotin esters; phospholipids, mixtures thereof, and the like.

Non-steroidal cosmetic soothing actives can be effective in preventing or treating inflammation of the skin. The soothing active enhances the skin appearance benefits of the present invention, e.g., such agents contribute to a more uniform and acceptable skin tone or color. Nonlimiting examples of cosmetic soothing agents include the following categories: propionic acid derivatives; acetic acid derivatives; fenamic acid derivatives; mixtures thereof and the like. Many of these cosmetic soothing actives are described in U.S. Pat. No. 4,985,459 to Sunshine et al., issued Jan. 15, 1991, incorporated by reference herein in its entirety.

Artificial tanning actives can help in simulating a natural suntan by increasing melanin in the skin or by producing the appearance of increased melanin in the skin. Nonlimiting examples of artificial tanning agents and accelerators include dihydroxyacetaone; tyrosine; tyrosine esters such as ethyl tyrosinate and glucose tyrosinate; mixtures thereof, and the like.

Skin lightening actives can actually decrease the amount of melanin in the skin or provide such an effect by other mechanisms. Nonlimiting examples of skin lightening actives useful herein include aloe extract, alpha-glyceryl-L-ascorbic acid, aminotyroxine, ammonium lactate, glycolic acid, hydroquinone, 4 hydroxyanisole, mixtures thereof, and the like.

Also useful herein are sunscreen actives. A wide variety of sunscreen agents are described in U.S. Pat. No. 5,087,445, to Haffey et al., issued Feb. 11, 1992; U.S. Pat. No. 5,073,372, to Turner et al., issued Dec. 17, 1991; U.S. Pat. No. 5,073,371, to Turner et al. issued Dec. 17, 1991; and Segarin, et al., at Chapter VIII, pages 189 et seq., of Cosmetics Science and Technology, all of which are incorporated herein by reference in their entirety. Nonlimiting examples of sunscreens which are useful in the compositions of the present invention are those selected from the group consisting of octyl methoxyl cinnamate (Parsol MCX) and butyl methoxy benzoylmethane (Parsol 1789), 2-ethylhexyl p-methoxycinnamate, 2-ethylhexyl N,N-dimethyl-p-aminobenzoate, p-aminobenzoic acid, 2-phenylbenzimidazole-5-sulfonic acid, oxybenzone, mixtures thereof, and the like.

Sebum stimulators can increase the production of sebum by the sebaceous glands. Nonlimiting examples of sebum stimulating actives include bryonolic acid, dehydroetiandrosterone (DHEA), orizanol, mixtures thereof, and the like.

Sebum inhibitors can decrease the production of sebum by the sebaceous glands. Nonlimiting examples of useful sebum inhibiting actives include aluminum hydroxy chloride, corticosteroids, dehydroacetic acid and its salts, dichlorophenyl imidazoldioxolan (available from Elubiol), mixtures thereof, and the like.

Also useful as actives in the present invention are protease inhibitors. Protease inhibitors can be divided into two general classes: the proteinases and the peptidases. Proteinases act on specific interior peptide bonds of proteins and peptidases act on peptide bonds adjacent to a free amino or carboxyl group on the end of a protein and thus cleave the protein from the outside. The protease inhibitors suitable for use in the present invention include, but are not limited to, proteinases such as serine proteases, metalloproteases, cysteine proteases, and aspartyl protease, and peptidases, such as carboxypepidases, dipeptidases and aminopepidases, mixtures thereof and the like.

Other useful as active ingredients in the present invention are skin tightening agents. Nonlimiting examples of skin tightening agents which are useful in the compositions of the present invention include monomers which can bind a polymer to the skin such as terpolymers of vinylpyrrolidone, (meth)acrylic acid and a hydrophobic monomer comprised of long chain alkyl (meth)acrylates, mixtures thereof, and the like.

Active ingredients in the present invention may also include anti-itch ingredients. Suitable examples of anti-itch ingredients which are useful in the compositions of the present invention include hydrocortisone, methdilizine and trimeprazineare, mixtures thereof, and the like.

Nonlimiting examples of hair growth inhibitors which are useful in the compositions of the present invention include 17 beta estradiol, anti angiogenic steroids, curcuma extract, cycloxygenase inhibitors, evening primrose oil, linoleic acid and the like. Suitable 5-alpha reductase inhibitors such as ethynylestradiol and, genistine mixtures thereof, and the like.

Nonlimiting examples of desquamating enzyme enhancers which are useful in the compositions of the present invention include alanine, aspartic acid, N methyl serine, serine, trimethyl glycine, mixtures thereof, and the like.

A nonlimiting example of an anti-glycation agent which is useful in the compositions of the present invention would be Amadorine (available from Barnet Products Distributor), and the like.

The invention will now be described in greater detail by way of the following non-limiting examples. The examples are for illustrative purposes only and not intended to limit the invention in any way. Physical test methods are described below:

Except in the operating and comparative examples, or where otherwise explicitly indicated, all numbers in this description indicating amounts or ratios of materials or conditions or reaction, physical properties of materials and/or use are to be understood as modified by the word “about”.

Where used in the specification, the term “comprising” is intended to include the presence of stated features, integers, steps, components, but not to preclude the presence or addition of one or more features, integers, steps, components or groups thereof.

All percentages in the specification and examples are intended to be by weight unless stated otherwise.

### EXAMPLE 1

A series of inventive examples T, D and comparative example H were made according to Table 1 using the procedure below in order to evaluate the effect of specific surfactants on various sensory properties. The sensory properties were determined via the trained panel testing using the procedure provided below and the results are summarized in Table 1. It was found that the inventive formulations provided overall superior sensory and stability properties compared to the comparative formulation.

TABLE 1

| | T (Inv.) | D (Inv.) | H (Comp.) |
|---|---|---|---|
| Component | % W/W | % W/W | % W/W |
| Water | 20.82 | 30.00 | 27 |
| C12 Disodium Sulfosuccinate | 6.00 | 6.00 | 6 |
| Cocamide MEA | | | 1.8 |
| Sodium Laureth Sulfate | | | 5.71 |
| Cocamidopropyl Betaine | | | 6.67 |
| Disodium cocamidoacetate | | | |
| Na Methyl Cocoyl Tauarate | 13.33 | | |
| Decyl Polyglucose | | | |
| Cocamido lauryl betaine | | | |
| Polyoxmer 407 (Pluracare F127) | | | 0.5 |
| Sodium cocoyl glycinate | | 5 | |
| Lauric Acid | 4.00 | 4.00 | 4.25 |
| Palmitic Acid | 0.70 | 0.70 | |
| Stearic Acid | 0.40 | 0.40 | |
| Sunflower Seed Oil | 11.00 | 11.00 | |
| Petrolatum | 1.00 | 1.00 | 1.25 |
| Glycerin | 40.00 | 40.00 | 30 |
| Sodium lactylate | | | 1.5 |
| Soybean oil | | | 12 |
| PEG 120 methyl glucose dioleate | | | 1.5 |
| Polyquaternium 10 | | | 1 |
| Lauryl Alcohol C12-14A | | | 1 |
| Polyquaternium 55 | 2.00 | 2.00 | |
| Methylparaben | 0.20 | 0.20 | 0.20 |
| Propylparaben | 0.10 | 0.10 | 0.10 |
| Phenoxyethanol | 0.40 | 0.40 | 0.40 |
| Disodium EDTA | 0.05 | 0.05 | 0.05 |
| Total | 100? | 100? | 100? |
| Rinse (a) | −0.5 | 0 | −3.75 |
| Lather type (b) | −1 | −2 | −1 |
| Skin Tightness (c) | 0.75 | 2 | −1.5 |
| Skin Feel (d) | 1.5 | 1 | 0.25 |

Notes:
(a) Panel test method no. 11
(b) Panel test method no. 6
(c) Panel test method no. 18
(d) Panel test method no. 12

### EXAMPLE 2

A series of inventive formulas D, T, 70, 72, 77, 84 were made and compared to comparative formulas 24, 25,26, 27, 28, 29, 30, 30A, 60, 69, according to Table 2 using the procedures described below.

The sensory properties were determined via the Laboratory Method—sensory aspect testing using the testing procedure provided below and the results are summarized in Table 2.

TABLE 2

| | D | T | 24 | 25 | 26 | 27 | 28 | 29 | 30 | 30A | 60 | 69 | 70 | 72 | 77 | 84 |
|---|---|---|---|---|---|---|---|---|---|---|---|---|---|---|---|---|
| | % | % | % | % | % | % | % | % | % | % | % | % | % | % | % | % |
| Component | W/W | W/W | W/W | W/W | W/W | W/W | W/W | W/W | W/W | W/W | W/W | W/W | W/W | W/W | W/W | W/W |
| Water | 30.75 | 28.75 | 30.45 | 30.75 | 30.35 | 30.65 | 28.05 | 23.75 | 34.75 | 36.75 | 34.75 | 33.75 | 40.75 | 40.95 | 39.95 | 39.75 |
| C12 Disodium | 8.00 | 8.00 | 8.00 | 8.00 | 8.00 | 8.00 | 8.00 | 8.00 | 4.00 | 4.00 | 6.00 | 6.00 | 8.00 | 8.00 | 8.00 | 8.00 |
| Sulfosuccinate | | | | | | | | | | | | | | | | |
| Taranol WSP | | 5.00 | | | | | | | | | | | | | | |
| Na Cocoyl | 3.00 | | 3.00 | 3.00 | 3.00 | 3.00 | 3.00 | 3.00 | 3.00 | 3.00 | 2.00 | 3.00 | 3.00 | 3.00 | 3.00 | 3.00 |
| Glycinate | | | | | | | | | | | | | | | | |
| Lauric Acid | 4.00 | 4.00 | 4.00 | 4.00 | 4.00 | 4.00 | 4.00 | 4.00 | 4.00 | 2.00 | 4.00 | 4.00 | 4.00 | 4.00 | 4.00 | 4.00 |
| Palmitic Acid | 0.70 | 0.70 | 0.70 | 0.70 | 0.70 | 0.70 | 0.70 | 0.70 | 0.70 | 0.70 | 0.70 | 0.70 | 0.70 | 0.70 | 0.70 | 0.70 |
| Stearic Acid | 0.40 | 0.40 | 0.40 | 0.40 | 0.40 | 0.40 | 0.40 | 0.40 | 0.40 | 0.40 | 0.40 | 0.40 | 0.40 | 0.40 | 0.40 | 0.40 |
| Sunflower Seed | 11.00 | 11.00 | 11.00 | 11.00 | 11.00 | 11.00 | 11.00 | 11.00 | 11.00 | 11.00 | 11.00 | 11.00 | 11.00 | 11.00 | 11.00 | 11.00 |
| Oil | | | | | | | | | | | | | | | | |
| Petrolatum | 1.00 | 1.00 | 1.00 | 1.00 | 1.00 | 1.00 | 3.70 | 8.00 | 1.00 | 1.00 | 1.00 | 1.00 | 1.00 | 1.00 | 2.00 | 2.00 |
| Glycerin | 40.00 | 40.00 | 40.00 | 40.00 | 40.00 | 40.00 | 40.00 | 40.00 | 40.00 | 40.00 | 40.00 | 40.00 | 30.00 | 30.00 | 30.00 | |
| Hydroxyethyl | | | | | | | | | | | | | | | | 30.00 |
| Urea | | | | | | | | | | | | | | | | |
| Polyquaternium | 0.40 | 0.40 | | | 0.40 | 0.40 | 0.40 | 0.40 | 0.40 | 0.40 | 0.40 | 0.40 | 0.40 | 0.20 | 0.20 | 0.40 |
| 55 | | | | | | | | | | | | | | | | |
| Jaguar C13S | | | 0.70 | 0.40 | 0.40 | 0.10 | | | | | | | | | | |
| Methylparaben | 0.20 | 0.20 | 0.20 | 0.20 | 0.20 | 0.20 | 0.20 | 0.20 | 0.20 | 0.20 | 0.20 | 0.20 | 0.20 | 0.20 | 0.20 | 0.20 |
| Propylparaben | 0.10 | 0.10 | 0.10 | 0.10 | 0.10 | 0.10 | 0.10 | 0.10 | 0.10 | 0.10 | 0.10 | 0.10 | 0.10 | 0.10 | 0.10 | 0.10 |
| Phenoxy- | 0.40 | 0.40 | 0.40 | 0.40 | 0.40 | 0.40 | 0.40 | 0.40 | 0.40 | 0.40 | 0.40 | 0.40 | 0.40 | 0.40 | 0.40 | 0.40 |
| ethanol | | | | | | | | | | | | | | | | |
| Disodium | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 | 0.05 |
| EDTA | | | | | | | | | | | | | | | | |
| Total | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 | 100.00 |
| Lather (e) | | | unaccep- | unaccep- | unaccep- | unaccep- | | unaccep- | unaccep- | | | unaccep- | accep- | accep- | | |
| | | | table | table | table | table | | table | table | | | table | table | table | | |
| Ease of | | | | | | | | | | | | | | | | |
| rinse (f) | | | | | | | | | | | | | | | | |
| Skin Feel (9) | | | unaccep- | unaccep- | unaccep- | unaccep- | | unaccep- | unaccep- | | | unaccep- | accep- | accep- | | accep- |
| | | | table | table | table | table | | table | table | | | table | table | table | | table |
| Thickness (h) | | | | | | | unaccep- | | | unaccep- | unaccep- | | accep- | accep- | accep- | accep- |
| | | | | | | | table | | | table | table | table | table | table | table | |

Notes:
(e): Laboratory Method - sensory aspect - Lather volume
(f): Laboratory Method - sensory aspect - Ease of rinse
(g) Laboratory Method - sensory aspect - Skin feel
(h): Laboratory Method - sensory aspect - Thickness (apparent viscosity)

### EXAMPLE 3

A series of inventive formulas were made according to Table 3 using the procedure below. The sensory properties were determined via the Laboratory Method—sensory aspect testing using the testing procedure provided below and the results are summarized in Table 3. It was found that the inventive formulations provided overall acceptable sensory and stability properties.

| Component | D1 | A | B | C | D |
|---|---|---|---|---|---|
| Water | 30.75 | 30.65 | 30.65 | | 30.75 |
| C12 Disodium | 8.00 | 8.00 | 8.00 | | 8.00 |
| Sulfosuccinate | | | | | |
| Sodium Methyl | | | | | |
| Cocoyl Taurate | | | | | |
| Na Cocoyl | 3.00 | 3.00 | 3.00 | | 3.00 |
| Glycinate | | | | | |
| Lauric Acid | 4.00 | 4.00 | 4.00 | | 4.00 |
| Palmitic Acid | 0.70 | 0.70 | 0.70 | | 0.70 |
| Stearic Acid | 0.40 | 0.40 | 0.40 | | 0.40 |
| Sunflower Seed Oil | 11.00 | 11.00 | 11.00 | | 11.00 |
| Petrolatum | 1.00 | 1.00 | 1.00 | | 1.00 |
| glycerin | | 0.00 | | | |
| propylene glycol | | | | | 40.00 |
| sorbitol | 40.00 | 40.00 | 40.00 | | 0.00 |
| Hydroxyethyl Urea | | | | | |
| Polyquaternium 55 | 0.40 | | | | 0.30 |
| polyquaternium 10 | | 0.50 | 0.50 | | |
| Jaguar C13S | | 0.00 | | | 0.10 |
| Methylparaben | 0.20 | 0.20 | 0.20 | | 0.20 |
| Propylparaben | 0.10 | 0.10 | 0.10 | | 0.10 |
| Phenoxyethanol | 0.40 | 0.40 | 0.40 | | 0.40 |
| Disodium EDTA | 0.05 | 0.05 | 0.05 | | 0.05 |
| Total | 100.00 | 100.00 | 100.00 | | 100.00 |
| lather | acceptable | acceptable | acceptable | | acceptable |
| Ease of rinse | acceptable | acceptable | acceptable | | acceptable |
| Skin feel | acceptable | acceptable | acceptable | | acceptable |
| thickness | acceptable | acceptable | acceptable | | acceptable |

### EXAMPLE 4

A series of inventive (D1) and comparative formulas (E, F, G, HJ, and H) were made according to Table 4 using the procedure below. The sensory properties were determined via the Laboratory Method—sensory testing using the testing procedure provided below and the results are summarized in Table 4. It was found that the inventive formulations provided overall superior sensory and stability properties compared to the comparative formulations.

TABLE 4

| Component | D1 | E | F | G | HJ | H |
|---|---|---|---|---|---|---|
| Water | 30.75 | 30.65 | 60.75 | 30.75 | 30.65 | 28.53 |
| C12 Disodium | 8.00 | | 8.00 | | 8.00 | 6.00 |
| Sulfosuccinate | | | | | | |
| Sodium Methyl | | | | | | |
| Cocyl Taurate | | | | | | |
| Sodium lauryl sulfate | | 8.00 | | 5.00 | | 6.00 |
| Cocofatty acid | | | | | | 1.80 |
| monoethanol amide | | | | | | |
| cocomidopropyl betaine | | 3.00 | | 3.00 | | 6.67 |
| Na Cocoyl Glycinate | 3.00 | | 3.00 | 3.00 | 3.00 | |
| Lauric Acid | 4.00 | 4.00 | 4.00 | 4.00 | 4.00 | 4.25 |
| Palmitic Acid | 0.70 | 0.70 | 0.70 | 0.70 | 0.70 | |
| Stearic Acid | 0.40 | 0.40 | 0.40 | 0.40 | 0.40 | |
| Sunflower Seed Oil | 11.00 | 11.00 | 11.00 | 11.00 | 11.00 | 11.00 |
| Petrolatum | 1.00 | 1.00 | 1.00 | 1.00 | 1.00 | 1.25 |
| glycerin | 40.00 | 40.00 | 10.00 | 40.00 | | 30.00 |
| propylene glycol | | | | | 40.00 | |
| sorbitol | | | | 0.00 | 0.00 | |
| Hydroxyethyl Urea | | | | | | |
| Polyquaternium 55 | 0.40 | | 0.40 | 0.40 | 0.00 | |
| polyquaternium 10 | | 0.50 | | | | 1.00 |
| Peg 120 | | | | | | 1.50 |
| Pationic 122A | | | | | | 1.25 |
| Jaguar C13S | | 0.00 | | | 0.50 | |
| Methylparaben | 0.20 | 0.20 | 0.20 | 0.20 | 0.20 | 0.20 |
| Propylparaben | 0.10 | 0.10 | 0.10 | 0.10 | 0.10 | 0.10 |
| Phenoxyethanol | 0.40 | 0.40 | 0.40 | 0.40 | 0.40 | 0.40 |
| Disodium EDTA | 0.05 | 0.05 | 0.05 | 0.05 | 0.05 | 0.05 |
| Total | 100.00 | 100.00 | 100.00 | 100.00 | 100.00 | 100.00 |
| Lather (e) | acceptable | Unacceptable | Unacceptable | Unacceptable | | Unacceptable |
| Ease of rinse (f) | acceptable | Unacceptable | | Unacceptable | Unacceptable | Unacceptable |
| Skin Feel (g) | acceptable | unacceptable | | | Unacceptable | |

Notes
(e): Laboratory Method - sensory aspect - Lather volume
(f): Laboratory Method - sensory aspect - Ease of rinse
(g) Laboratory Method - sensory aspect - Skin feel

Procedure for Sample Preparation for Examples 1 to 4
1 Weigh D1 water, Glycerin, EDTA-4Na and Glydant/Methyl paraben together in the main mixer and begin mixing and heating to 55 C.
2 Add Merquat 3330 and Merquat 550 or Styleze and water in a side tank—mix and then add to the main mixer when it reaches 55 C and hold for 15 min.
3 Heat to 80 C slowly. Then slowly add C12 Disodium Sulfosuccinate, and other surfactants to the main mixer and mix until completely dissolved. Add propyl paraben.
4 Predisperse TiO2 with water and when predispersed add to the main mixer and blend in.
5 Weigh fatty acids together and start heating and mixing until mixture reaches 80 C. Add glyceride oils and petrolatum and blend until uniform.
6 Add fatty acid blend to the Main mixer and mix for 15 min at 70-80′ C.
7 Cool down to 35° C.
8 Slowly add perfume and phenoxyethanol.
9 Let stand overnight before filling containers.
   Methods:
   Test Method for Sensory and Physical Properties

A panel is assembled of 10 persons and trained according to the following instructions and definitions. The assessments for each attribute of all the panelists are added and the average calculated and reported.

Panel Instructions and Definitions:

Wash your palm with unperfumed control soap (Ivory® soap available from Proctor and Gamble or its equivalent) and wet your face with water as required. Then provide individual assessments of test samples by selecting a value from the following numerical scales that most closely applies.

Before Use

1. Ease of Pouring from Container.

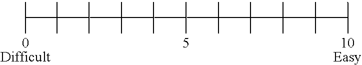

2. Thickness

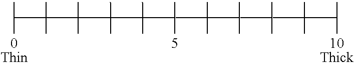

Pour the product (about 1 ml) onto the palm. Pour one teaspoonful of water onto the palm and dilute it 20 times with additional water.

Make a lather between hands by rubbing hands together 10 times to evaluate the ease of lathering.

Pour one teaspoonful of water onto palm and rub hands together again 30 times to evaluate amount of lather and rub hands together 10 times to evaluate the creaminess of lather. Then provide individual assessments of test samples by selecting a value from the following numerical scales that most closely applies.

Product in Palm

3. Ease of Diluting with Water

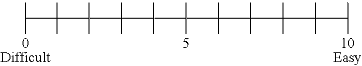

4. Ease of Lathering

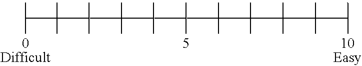

5. Lather Amount

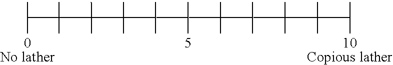

6. Creaminess of Lather or Lather Type

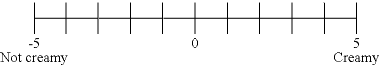

Start washing the face with the pre-lathered product until it feels it is time to rinse. Then provide individual assessments of test samples by selecting a value from the following numerical scales that most closely applies.

Product on Face

7. Ease of Spreading

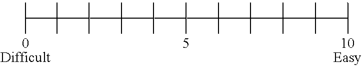

8. Stickiness

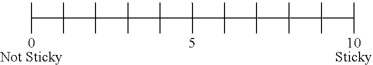

9. Slippery

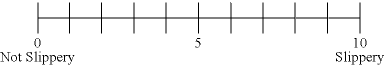

10. Lather Amount

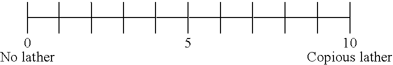

Rinse your face with water as needed until you feel it is time to dry it.

Then provide individual assessments of test samples by selecting a value from the following numerical scales that most closely applies.

While Rinsing

11. Ease of Rinse 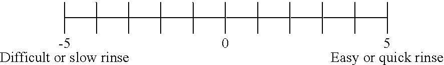
Wet Skin Feel

12. Slippery or Skin Feel

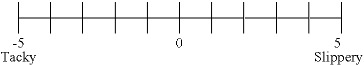

13. Dragginess

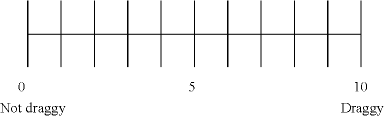

14. Cleanness

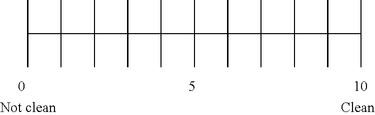

15. Freshness

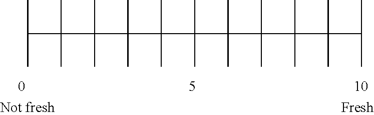

Dry the face with paper towel.

Wait for 2 minutes.

Then provide individual assessments of test samples by selecting a value from the following numerical scales that most closely applies.

Dry Skin Feel

16. Dryness

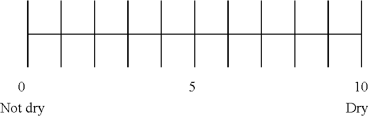

17. Smoothness

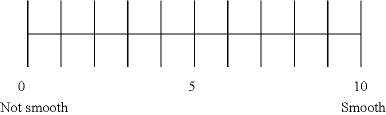

18. Tightness

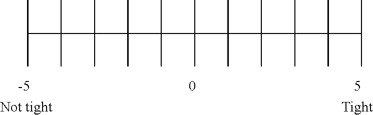

19. Pleasant

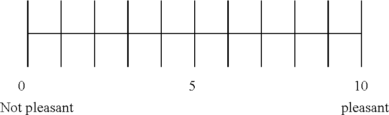

### Attribute Definitions for Panel Testing

Before Using the Product

1. Ease of Pouring
Difficult: It is difficult to dispense the product when pouring it from the container
Easy: It is easy to dispense the product when pouring it from the container.

2. Thickness
Thin: The appearance of the product is like water.
Thick: The appearance of the product is like honey.
   When Product is Used On the Palm

3. Ease of Diluting with Water
Difficult: The product is difficult to mix with water.
Easy: The product is easy to mix with water.

4. Ease of Lathering
Difficult: It takes a long time to lather between the hands.
Easy: It takes a short time to lather between the hands.

5. Amount of Lather
No lather: No lather appears when you rub the product between the hands
Copious of lather: Copious lather appears when you rub the product between the hands.

6. Creaminess of Lather
Not creamy: The appearance of the lather consists of large and/or coarse bubbles.
Creamy: The appearance of the lather appears creamy with fine uniformly sized bubbles.
   When Use the Product is Used On the Arms

7. Ease of Spreading the Product
Difficult: The product is difficult to distribute all over the forearm.
Easy: The product is easy to distribute all over the forearm.

8. Stickiness of the Product
Not sticky: It is easy to lift fingers from the skin.
Sticky: Resistance is felt when fingers are lifted from the skin.

9. Slippery
Not slippery: There is noticeable resistance felt when the skin is rubbed with fingertips
Slippery: There is no noticeable resistance felt when the skin is rubbed with fingertips

10. Amount of Lather
No lather: No lather appears when the arms are rubbed with the hands.
Copious lather: Copious lather appears when the arms are rubbed with the hands.
   When Rinsing the Arms

11. Ease of Rinse
Difficult: A great deal of water is required to remove product from the skin.
Easy: Only a small amount of water is required to remove product from the skin.
   Wet Skin Feel

12. Slippery
Not slippery: resistance to motion is felt when the skin is stroked with finger tips.
Slippery: no resistance to motion is felt when the skin is stroked with finger tips.

13. Dragginess
Not draggy: no resistance to motion is felt when the skin is stroked with finger tips.
Draggy: resistance to motion is felt when the skin is stroked with finger tips.

14. Cleanliness
Not clean: Sensation that dirt has not been completely removed from the skin.
Clean: Sensation that dirt has been completely removed from the skin.

15. Freshness
Not fresh: The feeling as if the skin is tired.
Fresh: The feeling as if the skin is on a mountaintop.
   Dry Skin Feel

16. Dryness
Not dry: The skin feels supple and moist.
Dry: The skin feels completely dry.

17. Smoothness
Not smooth: resistance to motion is felt when the skin is stroked with finger tips.
Smooth: the skin feels like silk when the skin is stroked with finger tips.

18. Tightness
Tight: The skin feels as if it has shrunk across the face.

19. Pleasant
Pleasant: A combined feeling of satisfaction and comfort is felt.
   Laboratory Method—Sensory Test Procedures
1) prewash hands with bar soap such as IVORY® bar soap available from Proctor and Gamble.
2) Dispense an approx. 18 mm diameter amount of product into hands.
3) Generate lather, as would a normal hand wash by rubbing wetted hands together for 30 seconds.
4) Rinse hands under warm water (approx. 35-40 C) until lather is sufficiently removed.
5) Pat dry with paper towel.
6) Sensory results are evaluated regarding lather generation, rinse time, inuse feel, and post use feel as described below.
   Lather Volume Sensory Aspect:

Is lather generated in a reasonable amount of time and is sufficient to user? Acceptable or Unacceptable.

Ease of Rinse Sensory Aspect:

Does the product rinse off skin with in a reasonable amount of time? Acceptable or Unacceptable.

Skin Feel Sensory Aspect:

Does the product when in use feel slippery or slimy? Acceptable or Unacceptable.

Doses the product after use does the skin feel tacky or not? Acceptable or Unacceptable.

Thickness (Apparent Viscosity) Sensory Aspect:

Can the product be dispensed from a flexible tube with an orifice of 5 mm in diameter without difficulty? Acceptable or Unacceptable.

Stability Method:

Samples may be stored at the following conditions and evaluated at the following evaluation points.

| | | | Evaluation |
|---|---|---|---|
| Condition | Time | Evaluations | Points |
| Approx. 25 C. | 12 weeks to 3 | Viscosity, Visual | Initial |
| | years | | 1 day |
| | | | 1, 2, 4, 8, 12 |
| | | | weeks, 3 years. |
| 40 C. | 12 weeks | Visual only | 1, 2, 4, 8, 12 |
| | | | weeks |
| 50 C. | 1 week | Viscosity, Visual | 1 week |
| −9 C./25 C. cycle | 3 cycles (6 days) | Viscosity, Visual | 1 week |
| (24 hours at each | | | |
| temp.) | | | |

Viscosity: Measured by the method indicated for each example
Visual evaluation: color, odor, and appearance

A sample is considered stable if its viscosity and visual evaluation do not change significantly (i.e. greater than 20% relative) from the initial measurements at all conditions.

T-Bar Viscosity Measurement:

Scope:

This method covers the measurement of the viscosity of a preferred embodiment of the invention that has an ordered liquid crystalline phase.

Apparatus:
Brookfield RVT Viscometer with Helipath Accessory;
Chuck, weight and closer assembly for T-bar attachment;
T-bar Spindle A;
Plastic cups diameter greater than 2.5 inches.

Procedure:
1. Verify that the viscometer and the helipath stand are level by referring to the bubble levels on the back of the instrument.
2. connect the chuck/closer/weight assembly to the Viscometer (Note the left-hand coupling threads).
3. Clean Spindle A with deionized water and pat dry with a Kimwipe sheet. Slide the spindle in the closer and tighten.
4. Set the rotational speed at 0.5 RPM. In case of a digital viscometer (DV) select the % mode and press autozero with the motor switch on.
5. Place the product in a plastic cup with inner diameter of greater than 2.5 inches. The height of the product in the cup should be at least 3 inches. The temperature of the product should be 25° C.
6. Lower the spindle into the product (˜¼ inches). Set the adjustable stops of the helipath stand so that the spindle does not touch the bottom of the plastic cup or come out of the sample.
7. Start the viscometer and allow the dial to make one or two revolutions before turning on the Helipath stand. Note the dial reading as the helipath stand passes the middle of its downward traverse.
8. Multiply the dial reading by a factor of 4,000 and report the viscosity reading in cps.

While this invention has been described with respect to particular embodiments thereof, it is apparent that numerous other forms and modifications of the invention will be obvious to those skilled in the art. The appended claims and this invention generally should be construed to cover all such obvious forms and modifications which are within the true spirit and scope of the present invention.

We claim:

1. A personal care cleansing composition having an ordered liquid crystalline structure, comprising:
a. at least about 20% by wt. of total polyol(s);
b. at least about 3% by wt. of total normal C6 to C16 alkyl monosulfosuccinate(s);
c. at least about 4% by wt. of vegetable derived glyceride oil(s);
d. at least about 0.1% by wt. of mineral oil(s);
e. a maximum of about 5% by wt. of soap;
f. at least about 15% by wt. of water;
g. an effective amount of an ordered liquid crystalline structurant for forming an ordered liquid crystalline phase in said composition;
h. a viscosity in the range of about 5,000 to 100,000,000 cps at 25 C using the t-bar method. and
i. wherein said composition has a lather type attribute of −1 or less and an ease of rinse attribute of −1 or greater according the Trained Panel Test.

2. The composition of claim 1 further comprising a skin tightness attribute of 0.5 or greater and a skin feel attribute of −0.2 or greater according to the Trained Panel Test.

3. The composition of claim 1 wherein total surfactant content is below a maximum of about 15% by wt.

4. The composition of claim 1 wherein the ratio of total polyol(s) to total lipophilic emollient(s) is in the range of about 1:40 to about 1:4.

5. The composition of claim 1 wherein the ratio of total lipophilic emollient(s) to total surfactant(s) is in the range of about 1:10 to about 10:1.

6. The composition of claim 1 wherein the ratio of total mono and disulfosuccinate(s) to total polyol(s) is in the range of about 1:4 to about 1:2.

7. The composition of claim 1 further comprising cationic polymer(s) in a concentration range of about 0.01% to 2.0% wt. %.

8. The composition of claim 1 wherein the polyol(s) is/are selected from glycerin, diglycerin, ethoxylated glycerin, propoxylated glycerin, propylene glycol, dipropylene glycol, polypropylene glycol Polyethylene glycol, sorbitol, hydroxylpropyl sorbitol, hexylene glycol, 1,3-butylene glycol and 1,2,6 hexanetriol and blends thereof.

9. The composition of claim 1 wherein the monosulfosuccinate(s) is/are selected from C10, C12, or C14 and blends thereof.

10. The composition of claim 1 wherein the vegetable derived glyceride oil(s) is/are selected from sunflower seed oil, soybean oil, jojoba oil, canola oil, coconut oil, peanut oil, or palm oil and blends thereof.

11. The composition of claim 1 wherein the mineral oil(s) is/are selected from petrolatum, mineral oil, heavy mineral oil, parafin wax, and blends thereof.

12. The composition of claim 1 wherein the cationic polymer(s) is/are selected from polyquaternium 55, polyquaternium 7, polyqaternium 39, polyquat 10, and blends thereof.

13. The composition of claim 1 wherein the composition has a lamellar structure.

14. The composition of claim 1 wherein the composition further comprises an effective concentration of an active ingredient selected from anti-acne actives, anti-wrinkle and anti-skin atrophy actives, skin barrier repair aids, cosmetic soothing aids, topical anesthetics, artificial tanning agents and accelerators, skin lightening actives, antimicrobial and antifungal actives, sunscreen actives, sebum stimulators, sebum inhibitors, antiperspirants, anti-glycation actives or mixtures thereof.
